(19) United States
(12) Patent Application Publication
(10) Pub. No.: US 2009/0165528 A1
(43) Pub. Date: Jul. 2, 2009

(54) Method and Apparatus for Gas Filter Testing

(75) Inventors: Jianquo Qian, St. Louis, MO (US); William Ronald Purdum, Maryland Heights, MO (US); Paul Brister, O'Fallon, MO (US)

Correspondence Address: KANG INTELLECTUAL PROPERTY LAW, LLC, 214 ELM STREET, SUITE 106, WASHINGTON, MO, 63090, US

(73) Assignee: CLEAN EARTH TECHNOLOGIES, LLC, St. Louis, MO (US)

(21) Appl. No.: 12/256,465

(22) Filed: Oct. 22, 2008

Related U.S. Application Data
(60) Provisional application No. 60/981,644, filed on Oct. 22, 2007.

(51) Int. Cl. G01N 33/00; G01N 7/00

(52) U.S. Cl. 73/1.06; 73/31.05

(57) ABSTRACT

A method is described to determine the remaining sorption capacity of activated-carbon-sorbent gas filters by measuring the breakthrough time for a test gas challenge to a test filter with known and controlled test conditions that include flow rate and temperature and the use of a calibration curve that has been established by prior testing of the test filter sorbent medium in calibration tests where the filter is exposed to a known quantity of gas surrogate for the challenge gases that the filter may sorb in service and then testing the test filter medium by challenge with a sparged gas that can be selectively detected to determine the breakthrough as a function of test gas. The apparatus using such a method comprises a portable system that includes a sparging test gas generator, a carrier gas system, a test filter canister holder, and a selective detector that can quantitatively monitor the test gas exiting the test filter canister. The method and apparatus can be used to determine the remaining sorption capacity of activated carbons filters, e.g., ASZM-TEDA carbon filters such as those used for building defense against chemical toxant attack, industrial accidents, and for tactical collective protection and for industrial ventilation and compliance with environmental regulations.

### CROSS-REFERENCE TO RELATED APPLICATIONS

This application claims the benefit of U.S. Application No. 60/981,644 filed Oct. 22, 2007, which is incorporated by reference in its entirety.

### STATEMENT REGARDING FEDERALLY SPONSORED RESEARCH

The development of the method and apparatus of this invention was supported, in part, by the Technical Support Working Group, an agency of the Federal Government, under contract W91CRB-06-C-5001. The Federal Government retains Government Purpose Rights, which include the right to use, modify, perform, display, release, or disclose technical data in whole or in part, in any manner or for any government purpose whatsoever, and to have or authorize others to do so in the performance of a Government Contract.

### APPENDIX

Not Applicable.

### BACKGROUND OF THE INVENTION

1. Field of the Invention

The invention relates to a method and apparatus to determine the remaining sorption capacity of activated-carbon-sorbent gas filters such as those used for building defense against chemical toxant attack, industrial accidents, and for tactical collective protection and for industrial ventilation and compliance with environmental regulations.

2. Related Art

Activated-carbon-sorbent gas filters are used in ventilation systems for tactical collective protection shelters and in building ventilation systems for defense against chemical toxant attack. Such filters are also used in building, industrial, submarine, and mining ventilation systems that may be susceptible to industrial accidents or the generation or release of toxic gas. In general, such filters are subject to rigorous testing for quality control and certification as a part of their manufacture, and they must meet demanding performance standards to ensure that filtered ventilation systems provide safe air for breathing.

Activated carbon filters, i.e., filters having carbon sorbent material that has been activated in the preparation of the carbon, remove undesirable vapors and gases from an air stream. Such filters act by sorption by physical adsorption/absorption, by chemi-sorption, and with the addition of one or more reactive or catalytic compounds in the sorbent material, by chemical reaction of the undesirable air constituents into species that are more readily sorbed or that are acceptable in the exhaust stream from the filter. An example of commonly found activated carbon filter medium with reactive/catalytic compounds embedded in the filter medium is ASZM-TEDA activated carbon (“ASZM carbon”), which contains metals such as copper, silver, zinc, molybdenum and triethylenediamine. This type of sorbent material is recommended by government guidance for chemical defense of ventilation systems (see for example, “Guidance for Filtration and Air-cleaning Systems to Protect Building Environments from Airborne Chemical. Biological, or Radiological Attacks” NIOSFI, CDC, DHHS Publication 2003-136, April, 2003).

The sorbent media for such filters are routinely tested prior to installation, and filter performance is commonly established by ‘destructive’ testing of a full scale filter or by extrapolation of a sub-scale filter. For this purpose, a lower toxicity surrogate is used, typically, as a test challenge in place of an actual threat chemical toxant as such threats may be a chemical warfare agent that is a controlled substance, e.g., because of a treaty agreement, or they may have high toxicity that poses a great hazard. Commonly, the selection of surrogate is based on a comparison of the physical properties of the surrogate with the threat chemical.

Various standard methods exist for filter or filter medium certification and quality control testing, but the prior art does not provide a practical “out of the laboratory” method for the determination of remaining filter sorption capacity testing filters once they are placed in service. Typically, for ASZM-carbon filters for chemical defense, samples from representative manufacturing batches of filter media are tested for their sorption capacity for three challenge toxic analytes prior to the loading of sorbent in the filter manufacturing process. In common practice, filter lifetime is estimated based on filter medium sorption capacity testing in ideal laboratory conditions, and then a recommended service lifetime that is shorter than the estimated lifetime is selected with consideration of the service conditions so that a filter replacement schedule can be established for routine maintenance. As a consequence of the desire for caution in determining the recommended service lifetime, filters are generally replaced although they may still have a large fraction of their absorption capacity. For large filter assemblies, such premature replacement comprises a considerable cost. On the other hand, in polluted environments or in tactical environments where multiple exposures to transient low level attack could occur, the capacity of a filter may diminish more rapidly, and so, the remaining sorption capacity at the end of the service life may be inadequate for sheltering requirements or safe use.

An accepted approach for the determination of remaining sorption capacity by testing of high value, large, activated-carbon filters after they have been placed in service, especially those used in heating ventilation and air conditioning (HVAC) systems, is to perform laboratory testing of a smaller filter that is located in a test filter canister that shares the air stream flowing into the larger filter, filter bank, or filter assembly, which will be referred to as the “main filter”. In such an arrangement, the test filter canister is situated in a parallel duct or bypass tube in which the input flow is adjusted so the axial air flow is the same as for the main filter. In this way, the ratio of the volumetric flow rate of input air and the volume of sorbent material is the same for both the test canister filter and the main filter. After a substantial fraction of the recommended service life or after a putative or confirmed exposure event, a decision to perform a test is made and the test canister filter is removed and sent to a laboratory for testing to determine the remaining sorption capacity. In the case of chemical defense filters, especially those used for government facilities, which may be in another country, the test canister filter is sent to a laboratory in the United States that is qualified and permitted to test items that may contain contamination by chemical warfare agents or other controlled substances. The handling, shipping, and testing require time that generally, amounts to several days or longer. It also leads to logistical challenges and risks posed by the potential for accidental release of items contaminated with hazardous materials.

In the prior art, the selection of test gas has been based on the similarity of the physical properties of the test gas to the anticipated challenge for the filter, i.e., on the properties of the test gas as a surrogate for a challenge gas or gases. However, unknown in the prior art and surprisingly discovered in our development of a test method is that such surrogate gas may susceptible to condensation in the filter media, which will lead to the inference of a greater filter sorption capacity than will occur in actual service. This can lead to a dangerous ‘over-rating’ of the filter service lifetime or a recommendation for filter replacement schedule that is over-optimistic.

The prior art does not provide a test method or readily portable apparatus that can determine the remaining sorption capacity of the main filter by testing the test canister filter in situ (i.e., in place) or in the immediate vicinity of the main filter, e.g., on or within the same facility (i.e., on-site). There are several challenges to performing such testing. One is the toxicity of the usual surrogate test gases, although the surrogates may be of much lower toxicity than chemical warfare agents. Another is the difficulty of making an accurate and precise quantitative measure outside of a laboratory with readily portable apparatus. Yet another is the difficulty of making a calibrated measurement in a situation where the filters (main and test) contain atmospheric pollution from the input air that may interfere with the measurement.

### SUMMARY OF THE INVENTION

A method is described to determine the remaining sorption capacity of activated-carbon-sorbent gas filters by measuring the breakthrough time for a test gas challenge to a test filter with known and controlled test conditions that include flow rate and temperature, and by the use of a calibration curve that has been established by prior testing of the test filter sorbent medium in calibration tests where the filter is exposed to a known quantity of gas surrogate for the challenge gases (“the contamination gas or gases”) that the filter may sorb in service, and then by testing the test filter medium by challenge with a sparged gas that can be selectively detected to determine the breakthrough as a function of test gas. The method includes selection criteria for test gas and the use of test operating conditions to avoid the condensation of the test gas in the test filter. Moreover, the method is well suited to use in a portable system that can test filters in situ or on site. The apparatus using such a method comprises a portable system that includes a sparging test gas generator, a carrier gas system, a test filter canister holder, and a selective detector that can quantitatively monitor the test gas exiting the test filter canister. The method and apparatus can be used to determine the remaining sorption capacity of activated carbons filters, e.g., ASZM-TEDA carbon filters such as those used for building defense against chemical toxant attack, industrial accidents, and for tactical collective protection and for industrial ventilation and compliance with environmental regulations.

The method and apparatus are used to determine the remaining sorption capacity of activated carbon filters, which may be subscale test filters in canisters that sample the input air flow to a larger filter, filter bank, or filter assembly. The method comprises the testing of the test filter by challenge with a selected test gas to determine the quantity of test gas that challenges the filter to obtain breakthrough and relating the breakthrough quantity with the remaining sorption capacity of the filter. The quantity of test gas challenge is a known quantity as a result of selection and control of the gas flow rate, temperature of the filter and certain parts of the apparatus, and the time duration of the challenge that leads to breakthrough. Once the measurement apparatus has been calibrated with known operating conditions, the breakthrough time is related to the remaining sorption capacity. For a range of flow rates and operating temperature, the breakthrough time is a linear function of the remaining sorption capacity. The challenge test gas is generated by gas sparging from a liquid reservoir, and the sparging gas mixture (SG), which includes the test gas component, is carried by a non-reactive, non-sorbed carrier gas to the test filter.

The method provides a set of selection criteria for a suitable test gas. The test gas is selected to be readily sorbed by the activated carbon but not readily decomposed by catalytic or reactive materials embedded in the carbon, and to be readily monitored and measured by a chemically selective detector. The test gas is further selected for vapor pressure, boiling point, molecular weight, shipping risk, and toxicity so that condensation in the filter is avoided and for practical use in a portable system. The method includes the selection of the test gas, a calibration procedure so that in situ on on-site measurement can be performed with readily portable, calibrated equipment, and especially to enable use of the test apparatus by a test operator with minimal training, the insertion of the test filter into the gas flow circuit of the test apparatus, the determination of the breakthrough time, and the use of the calibration curve to determine the remaining sorption capacity of the test filter and its correlation to a “main filter”. Still further, the method provides a criterion for the selection of the test operating temperature so that condensation of the test gas in the sorbent material is avoided. Such condensation is undesirable because it can alter the relation between breakthrough time and remaining sorption capacity for a constant flow rate challenge of test gas.

The method results in the determination of remaining sorption capacity without altering the content of sorbed gases already in the test filter medium. As a consequence, contaminant gases sorbed by the filter sorbent prior to the test are not displaced out of the filter medium.

Using the selection criteria, suitable test gases are found to include halogenated volatile gases. These gases are readily monitored and measured by well-developed and available detectors, e.g., electron-capture detectors and related devices, also by a dry electrolytic conductivity detector or DELCD. An example of a test gas suitable for testing at or near room temperature is dichloromethane.

In one preferred embodiment the method and apparatus may be used to determine the remaining sorption capacity of a test canister filter in situ by attachment of the test apparatus to the input and output flow of the test canister with suitable valves to control the flow. In another preferred embodiment, the method and apparatus may be used be used to determine the remaining sorption capacity of a test canister filter on site or in any convenient location by removal of the test canister filter and its insertion into a canister filter holder that is part of the test apparatus. The apparatus for these embodiments is readily portable and is pre-calibrated for the particular sorbent material.

### BRIEF DESCRIPTION OF THE DRAWINGS

The present invention will become more fully understood from the detailed description and the accompanying drawings, wherein:

FIG. 1 illustrates the flow diagram of the test method of the present invention.

FIG. 2 illustrates a flow chart demonstrating the use of the criteria for test gas selection.

FIG. 3 illustrates representative data of an ECD detector signal as a function of time to show that the breakthrough time is seen as a sharp rise in the detector signal approximately 50 to 80 minutes after the start of the controlled and regulated flow of test gas into the test filter.

FIG. 4 illustrates the breakthrough time as a function of percentage of “fresh carbon”, i.e., the remaining sorption capacity of the filter, in a calibration test.

FIG. 5 is a schematic drawing of an embodiment of the apparatus for determining the remaining sorption capacity.

FIG. 6 illustrates a comparison of the breakthrough time for DMMP and 2 CEME, which are used as surrogates for contamination in the generation of calibration curves.

FIG. 7(a) illustrates ECD detector response in a test sequence that shows no significant condensation of the test gas (2-chloropropane) in the test filter medium.

FIG. 7(b) illustrates ECD detector response in a test that shows significant condensation of the test gas (2-chloroethyl methyl ether) in the filter medium.

FIG. 8 illustrates that the detector response increases and is proportional to the sparging gas flow rate.

FIG. 9 illustrates a cross section view of the test canister holder of the present invention.

FIG. 10 is an elevated view of the base/bottom of an open test canister holder.

FIG. 11 is an elevated view of an open test canister holder with a test canister in place.

FIG. 12 is an elevated view of a closed test canister holder of the present invention.

FIG. 13 is an elevated view of a temperature controlled enclosure that contains a canister holder and a sparging gas generator.

FIG. 14 is an elevated view of a portable system comprising the apparatus of the present invention.

FIG. 15 is a front view of a portable system comprising the apparatus of the present invention as packaged for field use.

### DETAILED DESCRIPTION OF THE INVENTION

The method employs a test gas 64 that exhibits reversible binding affinity for the carbon filter element 108, instrumentation 66 to generate the test gas, and a highly sensitive gas analyzer 88 to determine test gas concentration for comparison with a calibration test gas breakthrough time versus remaining sorption capacity, i.e., “remaining filter-life” graph with an expected error rate of less than 5%.

ASZM-TEDA-carbon is the U.S. military designation for a recent version of activated carbon impregnated with copper, silver, zinc, and molybdenum salts. ASZM-TEDA filters are widely used in building FIVAC systems, including many government buildings. These filters are expensive, and managing building operations requires an assessment of both filter performance and remaining life before costly replacement filters will be needed. Ensuring appropriate protection of building occupants requires filter testing that can in itself be expensive and time-consuming. The method and apparatus of this invention provides facilities managers with less expense and a rapid test method for determining remaining filter life.

The test method is based on the comparative physical adsorption capacities of fresh and contaminated activated carbons for a test gas 64 using breakthrough time as a metric. Although the retention time may be used to evaluate the physical adsorption capacity, the physical property of the test gas restricts its use because this testing requires the increase in the operation temperature, and the temperature increase may cause the change in the original condition of the activated carbon, which will be contaminated by environmental pollution and may have contain contaminants from prior transient exposures.

The relationship between the sorption capacity and breakthrough time is used to evaluate the sorption lifetime of an activated carbon filter. This is accomplished by using a test gas 64 to determine the remaining sorption capacity of the activated carbon without significant change in the original condition of the activated carbon, e.g., without displacement of prior contamination out of the filter.

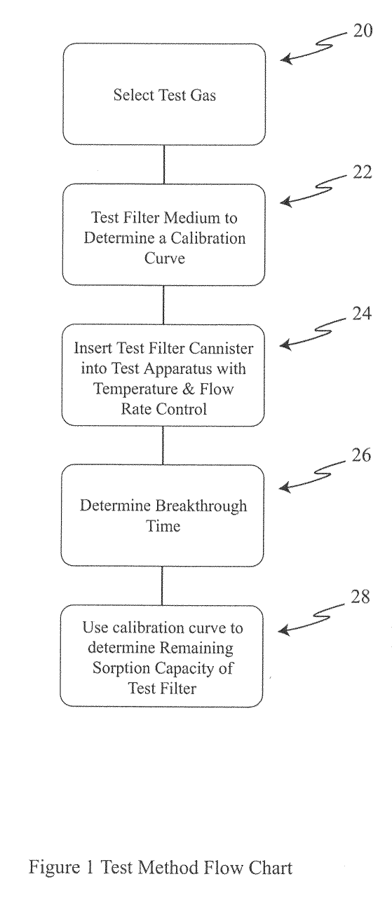

Test Method: The steps of the test method are shown in FIG. 1. The test method comprises the steps of:

selecting the test gas 20;

performing a calibration procedure to generate a calibration graph (curve) 22;

inserting the test filter 108 into the gas flow circuit 58 of the test apparatus 24 with temperature control of the sparging gas generator 64, gas flow circuit 58, and test filter 108;

determining the breakthrough time 96;

and using the calibration curve 28 to determine the remaining sorption capacity 98 of the test filter 108;

and, optionally, correlating the results for the test canister to a “main filter” 100.

Still further, the method provides a criterion for the selection of the test operating temperature so that condensation of the test gas 20 in the sorbent material is avoided. Such condensation is undesirable because it can alter the relation between breakthrough time and remaining sorption capacity for a constant flow rate challenge of test gas.

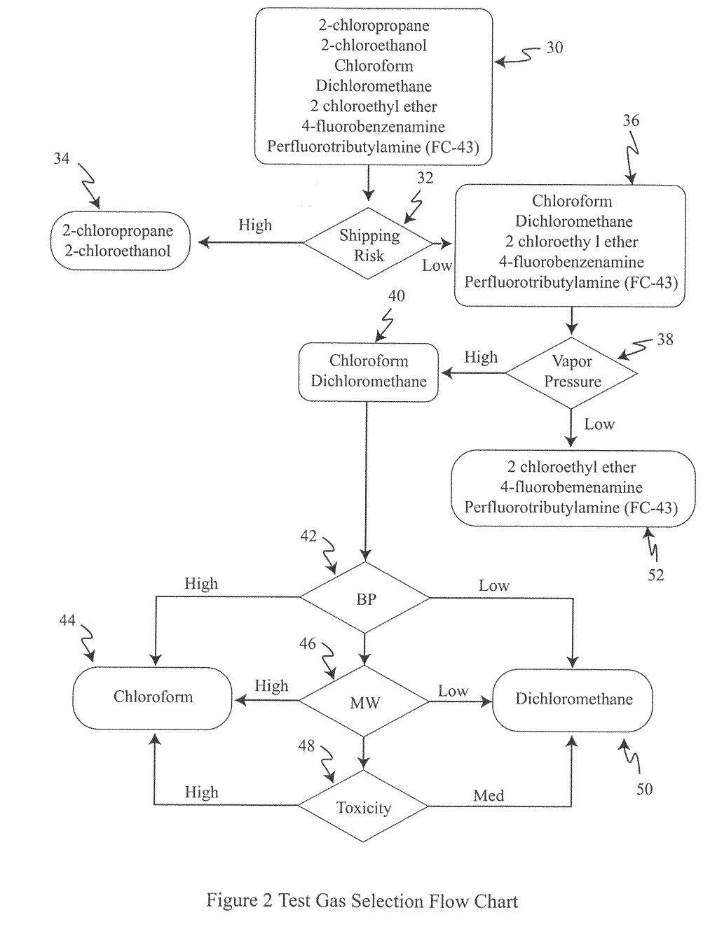

Test Gas Selection: Test gas 20 selection was made using a flow chart (FIG. 2) based on criteria of descending importance for the “on site” measurement application, whereby system precision and accuracy was given the greatest consideration since safety factors can be mitigated with effective engineering controls. At a minimum, the test gas should have relatively high vapor pressure, and a special functional group for selective detection. If an electron capture device is used as the detector, the functional group must be halogenated. If a dry electrolytic conductivity detector is used, the test gas must be halogenated but not fluorinated. In a preferred embodiment, the test gas 20 is chlorinated and/or brominated. Candidate test gases were also selected for relatively low toxicity, non-flammability, commercial availability, and low shipping risk. Other considerations were adsorption and desorption properties, condensation, predicted breakthrough times and detection interferences.

Table A shows the candidate test gases along with the relevant information used for selection.

TABLE A
Candidate Test Gases

| | | | Vapor | | | | |
|---|---|---|---|---|---|---|---|
| | Boiling | Molecular | Pressure | | Flammability | | |
| | Point | Weight | (Torr) @ | Relative | (flash point) | Commercial | Shipping |
| Test Gas | (BP) ° C. | (Dal) | 25° C. | Toxicity | ° C. | Availability | Risk |
| 2-Chloropropane | 35 | 78.5 | 350 | Low | −35 | Yes | High |
| 2-Chloroethanol | 129 | 80.5 | 5 | High | 60 | Yes | High |
| Chloroform | 61.7 | 119.4 | 159 | High | None | Yes | Low |
| Dichloromethane | 39.8 | 84.9 | 350 | Medium | None | Yes | Low |
| 2-Chloroethyl ether | 65-67 | 143 | 1.55 | High | 55 | Yes | Medium |
| 4-Fluoro- | 187 | 111.1 | 1 | N/A | 67 | Yes | N/A |
| benzenamine | | | | | | | |
| Perfluoro- | 155 | 671.1 | 0.552 | N/A | Non- | Yes | N/A |
| tributulamine | | | | | combustible | (expensive) | |
| (FC-43) | | | | | | | |

N/A = not applicable

Several candidate test gases were tested. These included 2-Chloropropane, 2-chloroethanol, chloroform, dichloromethane, 2-chloroethyl ether, 4-fluorobenzenamine, 30, 32. In a preferred embodiment, the test gas is dichloromethane 50.

Test gases meet the selective detection requirement to avoid the potential interferences from contaminants eluted out of the activated carbon during the testing by carrier gas. Condensation of the test gas should be avoided during the testing. To avoid condensation, the boiling point (BP) of the test gas should be close to the test operation temperature. Selection of a test gas 20 with a boiling point that is greater than the test operation temperature makes for readily controlled sparging of the test gas. However, if the boiling point of the test gas is too high, then condensation becomes likely. For operation in an ambient temperature range of 0 to 30° C., and when dichloromethane (BP=39.8° C.) is selected as the test gas, then the test operation temperature can be selected to be about 30° C. Further, if a test gas with a boiling point that is very much greater than the test operation temperature is selected, then the vapor pressure will be low, and the test may take a long time, for example several hours vs. about hour, which is typical for dichloromethane at 30° C. In any event, the calibration curve for the specific test operation temperature must be used. Once the test gas is selected, the calibration curve is generated at the desired operating temperature.

Calibration Procedure: The calibration is setup by following steps.

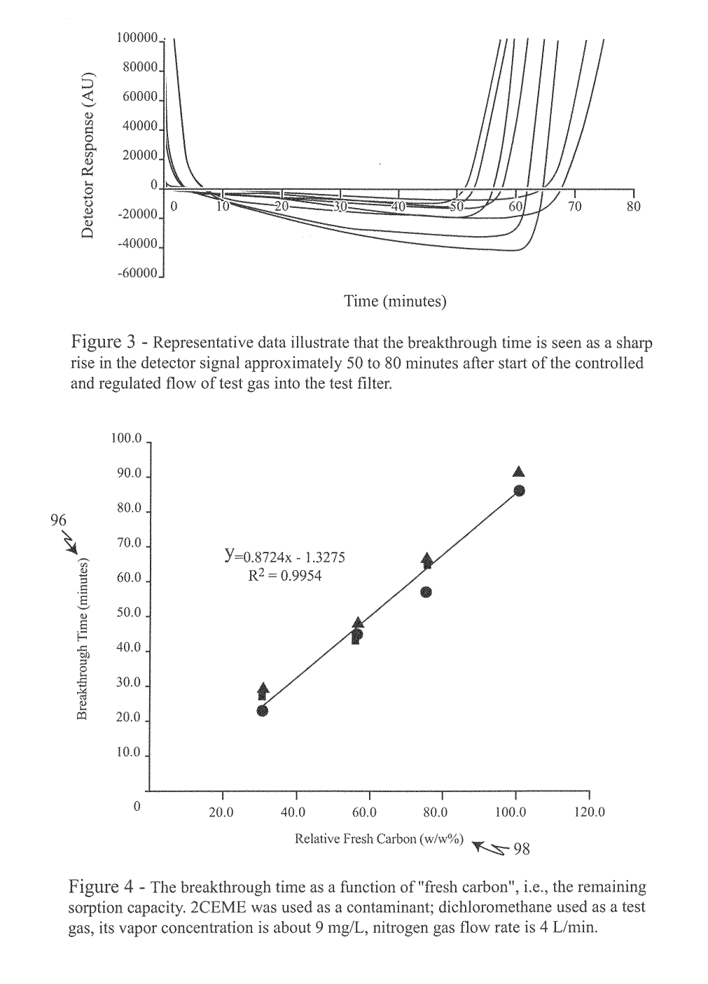

1. The step 20 of adding a Mock Contaminant gas 64 to the activated carbon by using a low toxicity organic compound. In preferred embodiments, the Mock Contaminant gas 64 is selected from the group consisting of DMMP and 2-chloroethyl methyl ether. In another preferred embodiment, the Mock Contaminant gas 64 comprises a mixture of both of these compounds. The Mock Contaminant gas 20 is used to saturate a portion of the activated carbon.
2. The step 26 of generating a curve of breakthrough time vs fraction of fresh carbon by the step 22 of mixing the Mock Contaminant gas 64 saturated carbon with the fresh activated carbon and packing 24 the mixture into an empty canister 108 with a selected ratio of fresh and saturated carbon and measuring the breakthrough time 96, so that the breakthrough time 96 vs. fraction of fresh carbon 98 can be determined, and then repeating the mixing, packing, and measuring for a set of various ratios of fresh and saturated carbon.
3. The step 28 of generating one or more calibration graphs (curves) under one or more selected test operation conditions, such as temperature, carrier gas flow rate, sparging gas flow rate, breakthrough time range, etc. FIG. 3 illustrates representative data of an ECD detector signal as a function of time to show that the breakthrough time is seen as a sharp rise in the detector signal approximately 50 to 80 minutes after the start of the controlled and regulated flow of test gas into the test filter.

FIG. 4 illustrates the breakthrough time 96 as a function of percentage of “fresh carbon” 98, i.e., the remaining sorption capacity 98 of the filter, in a calibration test. This is a typical calibration curve.

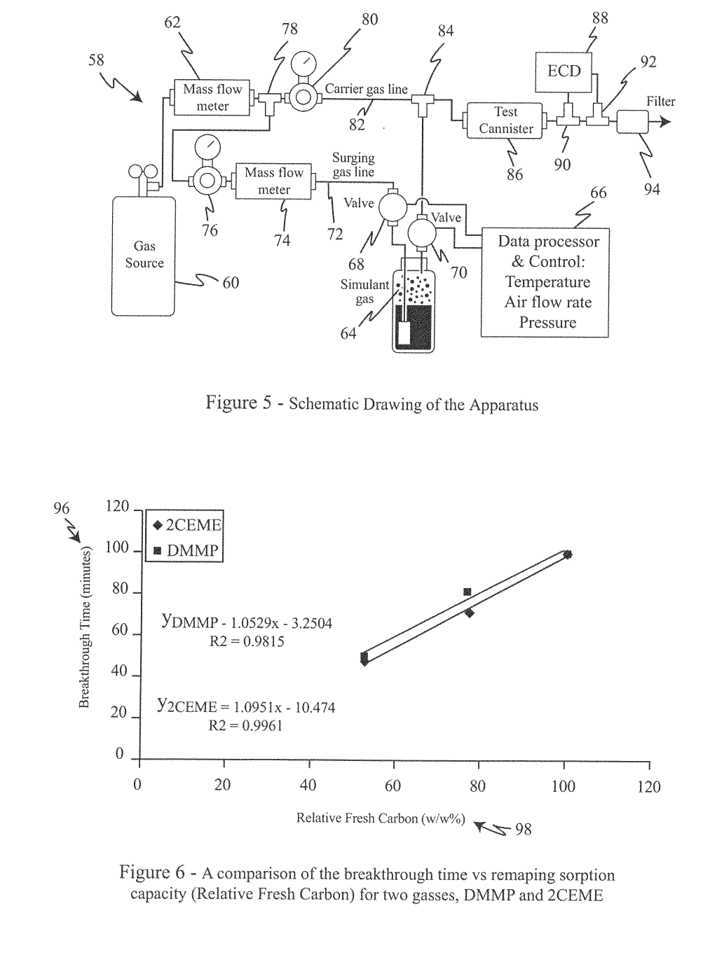

FIG. 5 is a schematic drawing of an embodiment of the apparatus for determining the remaining sorption capacity 98. The carrier gas 60 and sparging gas 60 were mixed in a Tee mixer 84 before getting into the canister 86. After the canister 86, the mixed gas was split by a SS Tee 90. The split ratio was controlled by a 0.01″ ID SS restriction tubing to make sure that the gas into the detector 88 was about 20 mL/min. All waste gas went out by passing an activated carbon filter 94 to remove most of contaminants in the waste gas

Mock Contamination (MC) Selection: Contaminants in the environment are very heterogeneous and represent a range of molecular sizes and vapor pressures. For this study two Mock Contaminants with significantly different molecular weight, size, and vapor pressure were chosen. These are dimethyl methylphosphonate (DMMP) and 2-chloroethyl methyl ether (2CEME, Table B).

TABLE B
Candidate Mock Contaminant Gases

| | | | Vapor | | | | |
|---|---|---|---|---|---|---|---|
| | Boiling | Molecular | Pressure | | Flammability | | |
| | Point | Weight | (Torr) @ | Relative | (flash point) | Commercial | Shipping |
| Test Gas | (BP) ° C. | (Dal) | 25° C. | Toxicity | ° C. | Availability | Risk |
| Dimethyl | 181 | 124.1 | 0.962 | N/A | | Yes | Low |
| methylphosphonate | | | | | | | |
| (DMMP) | | | | | | | |
| 2-Chloroethyl | 89-90 | 94.5 | 10 | N/A | 15 | Yes | N/A |
| methyl ether | | | | | | | |
| (2CEME) | | | | | | | |

N/A = not applicable

DMMP was selected because it is presently used for QA/QC by activated carbon manufacturers and it is the test gas 64 in the current protocol used by ECBC for the determination of breakthrough time 96 in ASZM-TEDA-activated carbon canisters. Physical sorption of a molecule to the ASZM-TEDA-activated carbon is dependent on the effective sorption Hole Size and Hole Number of the activated carbon and temperature. The Hole Size is related to the molecular size of the contaminant, the Hole Number is related to how many molecules can be sorbed, and the temperature is related to the contaminant vapor pressure. If two chemicals with significantly different molecular sizes and vapor pressures are both effectively sorbed by a given type of activated carbon, then the activated carbon is considered to have the capacity to adsorb a wide range of chemicals.

The test results indicate that the tested ASZM-TEDA-carbon has similar adsorption capacity for DMMP and 2CEME if counting with mole absorption rate (FIG. 6). The loading rate is about 12.5-13% (w/w). In this case, DMMP saturated activated carbon appeared to have slightly more absorption capacity available than the 2CEME saturated activated carbon because under the similar weight percentage loading, DMMP has a smaller molecular number loading rate. Table B shows DMMP has greater molecular weight than 2CEME. With the assumption of the activated carbon having similar absorption capacity (M/g % unit) with different Mock Contaminants within a range of molecular weights, and under the same weight loading rate, the greater molecular weight (less molecular numbers) should correspond with more available absorption capacity. When comparing test gases and evaluating the absorption capacity of the filter medium for the test gas, a longer breakthrough time should result for the greater molecular weight test gas loaded activated carbon. Our test results are in good agreement with this assumption. FIG. 6 shows that the breakthrough time is longer when using DMMP than when using 2CEME as the Mock Contaminant when the same weight loading rate is used.

In FIG. 6, the Mock Contaminant saturated activated carbon was made at a relatively high temperature and over a long time period so to achieve absorption equilibrium. For example, 13% of DMMP was added to the fresh activated carbon in glass bottle, then completely closed the bottle and put it to an oven for 5 days. The oven temperature was set at 110° C., and the bottle was shaken to mix DMMP and the carbon well at least 5 times per day during the 5 days. 2CEME saturated was carbon made at in similar saturation conditions.

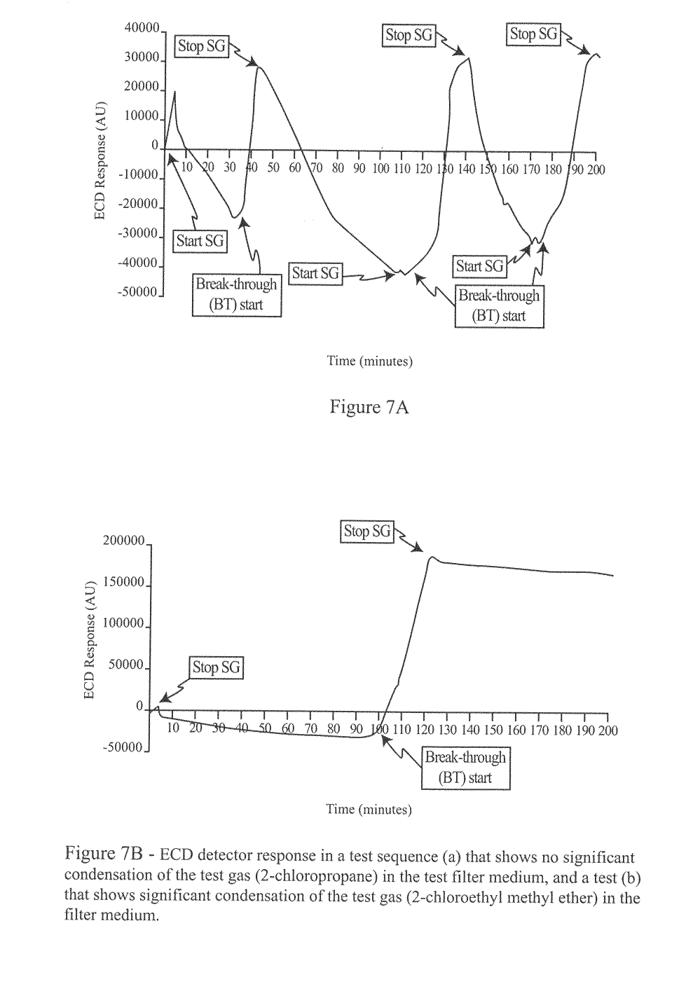

FIGS. 7(a) and 7(b) show the detector signal as a function of time for a sequence of sparging cycles. The ECD detector 88 response in a test sequence in FIG. 7(a) shows no significant condensation of the test gas (2-chloropropane) in the test filter medium 108. In FIG. 7(b), the ECD detector 88 response in a test shows significant condensation of the test gas (2-chloroethyl methyl ether) in the filter medium 108. In FIG. 7(a) when sparging is stopped, the detector 88 response decreased quickly, which means no significant condensation occurred. In this case the test gas is 2-chloropropane, which has a boiling point BP=35° C. In FIG. 7(b) with the test gas being 2-chloroethyl methyl ether, which has a boiling point BP=89° C., when the sparging stopped at ˜120 minutes (and after breakthrough 96 has occurred at ˜98 minutes), the detector 88 response decreased very slowly, with recovery time possibly being 24 hours or more to return to a normal level. This indicates that test gas condensation occurred and shows that a low boiling point test gas is preferable.

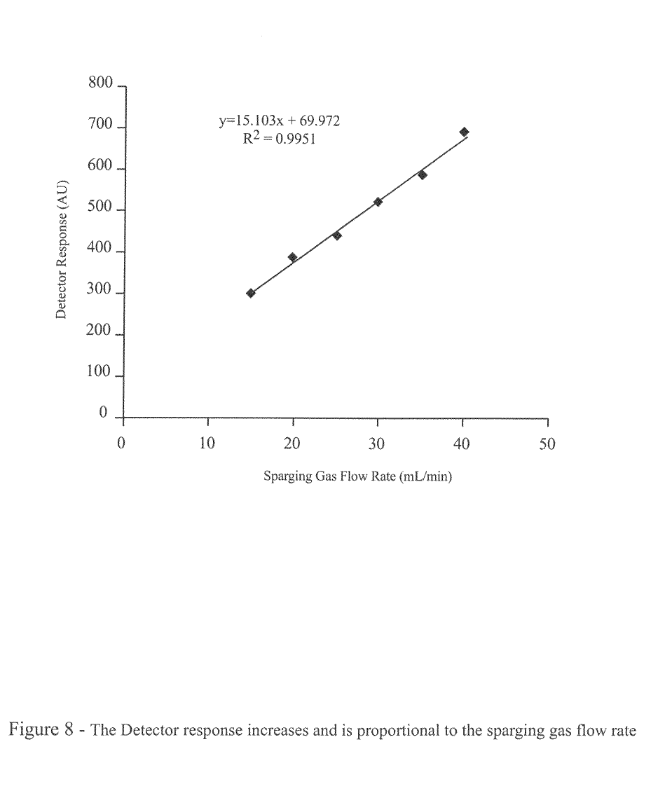

FIG. 8 illustrates that the detector 88 response increases and is proportional to the sparging gas flow rate as measured by mass flow meter 74.

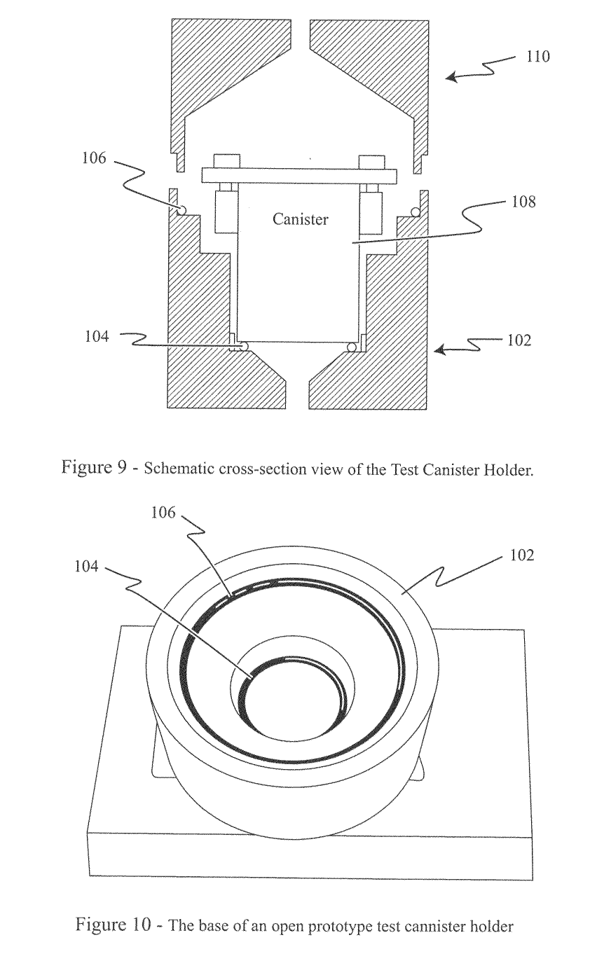

FIG. 9 illustrates a cross section view of the test canister holder 86. Using two O-rings, 104 and 106, respectively, the canister holder 86 provides a gas tight seal to the test filter canister 108. In a preferred embodiment, the canister holder 86 allows for the fast installation by hand without requesting special tools and without changing the original status of the canister 86.

FIG. 10 is an elevated view of the base/bottom 102 of an open test canister holder 86.

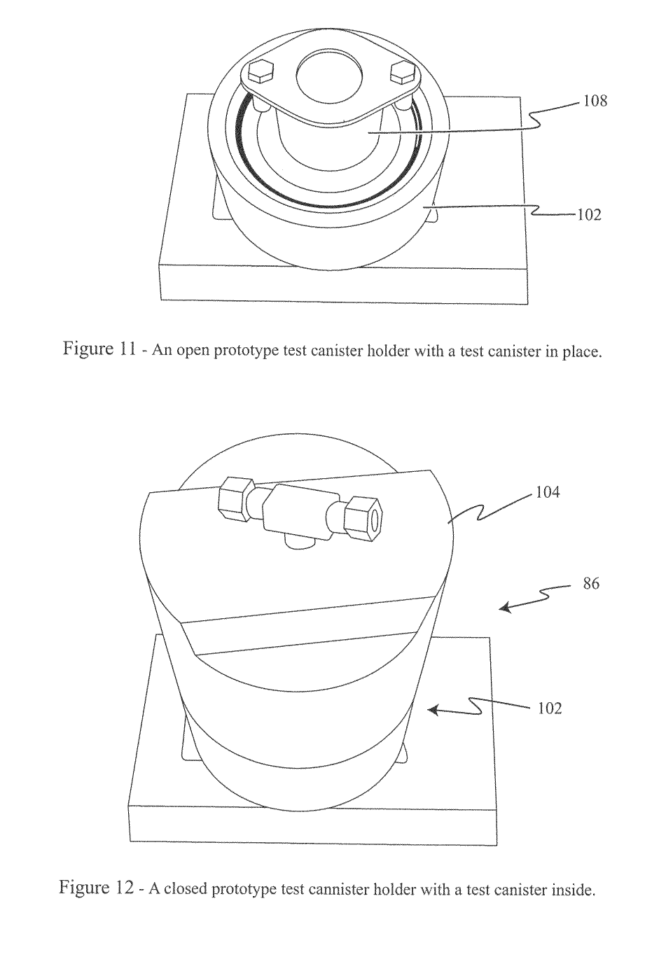

FIG. 11 is an elevated view of an open test canister holder 86 with a test canister 108 in place.

FIG. 12 is an elevated view of a closed test canister holder 86 of the present invention.

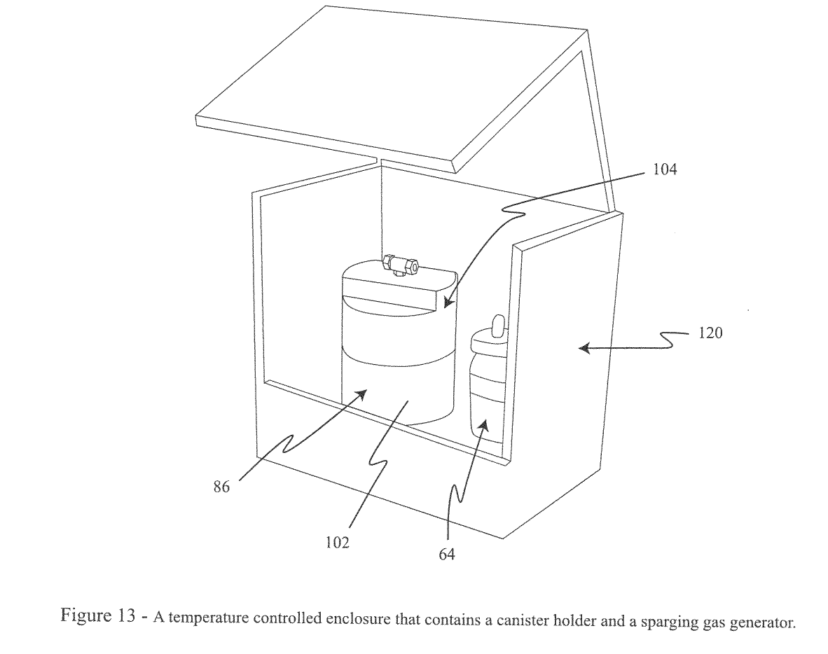

FIG. 13 is an elevated view of a temperature controlled enclosure, shown generally at 120, that contains a canister holder 86 and a sparging gas generator 60.

The standard deviation of the test system was shown to be 10% or less. To meet the requirement of less than 5%, the training system will include a well-controlled SG generator 60 temperature and sparging gas flow rate as measured by mass flow meter 74.

A Portable ASZM-Carbon HVAC Filter Test Kit can be used to evaluate the remaining lifetime of activated carbon filled in the ASZM-Carbon HVAC Filter by testing its canister. Its major parts are:

a) Carrier gas supply, sparging gas 60 using the same gas.

b) Carrier gas and sparging gas 60 adjusting and stabilizing system.

c) Test gas 64 generation by a sparging gas system with a liquid reservoir.

d) Canister connection system that has a canister holder 86 that permits easy emplacement of a test canister 108 or connection means so that an isolated test canister 108 can be inserted into the test gas/carrier gas flow system of the test apparatus.

e) Temperature control of the test canister and test gas generator system.

f) An optional and stable gas-split system

g) a selective detector 88 of the test gas, and

h) a data acquisition and instrument control system 66, which includes operating software and a computer with a display.

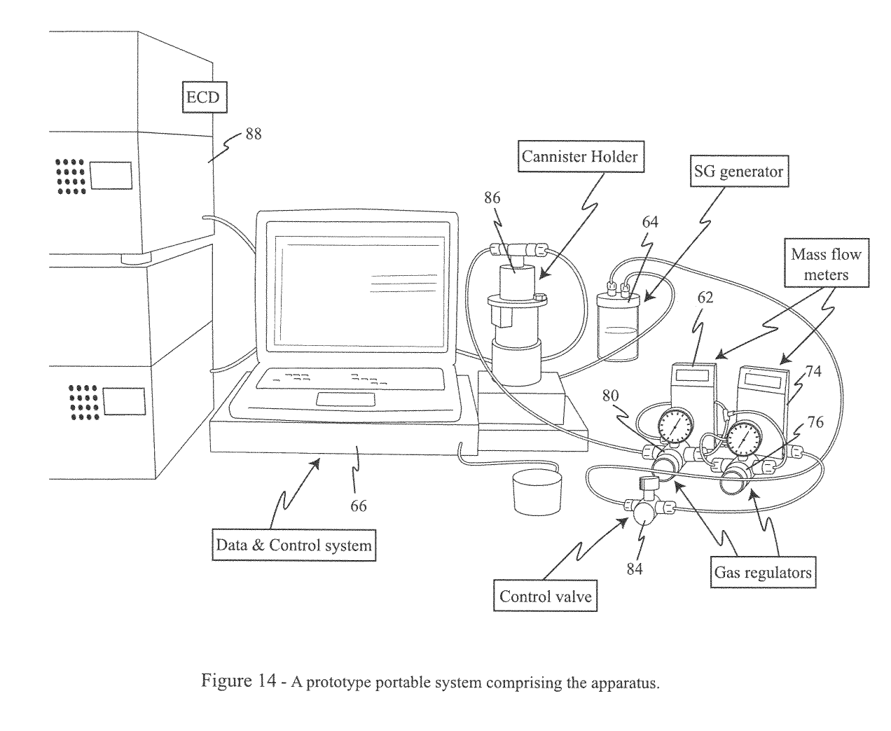

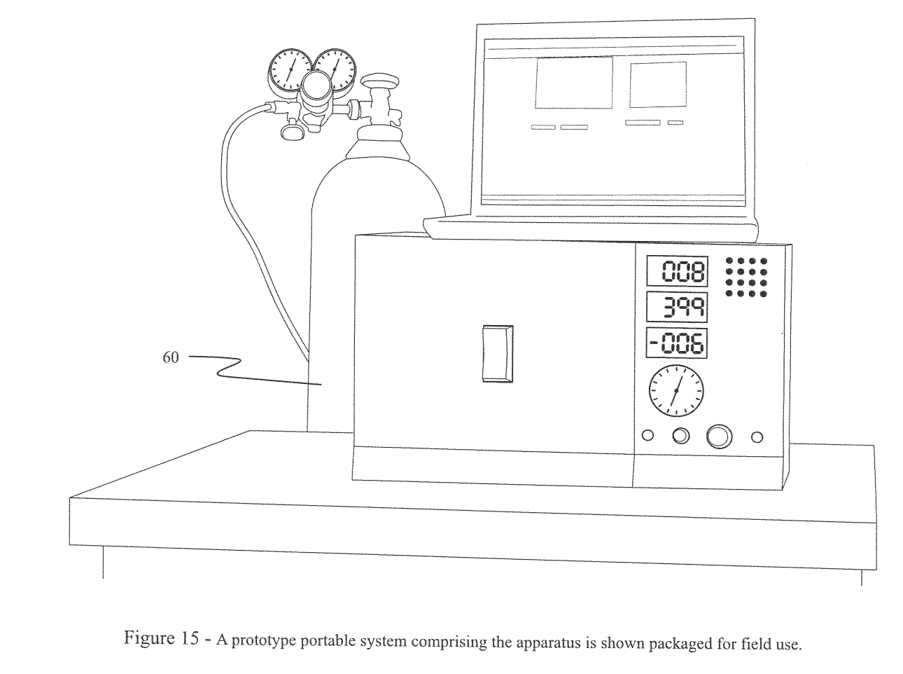

A portable system comprising the apparatus is shown in FIG. 14. A portable system comprising the apparatus is shown packaged for field use is shown in FIG. 15. This system has the following component features:

Carrier Gas (CG) 60: High purity liquid nitrogen and ‘zero’ air are used in this prototype. The carrier gas 60 is controlled by a gas regulator 80 with a flow rate of 4.0 L/minute. Change in the gas regulator 80 setting can change the carrier flow rate based on the testing requests.

Sparging Gas (SG) 60: High purity liquid nitrogen and ‘zero’ air are used in this prototype. The sparging gas 60 is used for the generation of the test gas 64. Because its flow rate is very low compared to the carrier gas 60 flow rate, a gas regulator 76 and stainless steel tubing are used to control the flow rate. The flow rates are from 10 to 100 ml/minute, which depends on the test gas vapor pressure. For higher vapor pressure, a lower sparging gas flow rate should be used. The flow rate should be controlled to meet two requirements: (1) Avoidance of test gas condensation. The test gas condensation may negatively affect the evaluation of the breakthrough time 96 that will result in an incorrect evaluation of filter remaining lifetime. (2) To optimize the breakthrough time 96 so that the testing time is short and the relative standard deviation is low. If the flow rate is too low, the breakthrough time 96 will be too long. If the flow rate is too high, the relative standard deviation will be high.

Gas Regulators, 76 and 78: The gas regulators (operating range of 0-100 psi), 76, and 78, are used to control each gas flow rate and to supply a stable gas flow. To compensate for a large difference between the carrier gas flow rate and the sparging gas flow rate, restrictive stainless steel tubing was used in the sparging system to supply very low and very stable sparging gas flow.

Mass Flow Meters, 62 and 74: The mass flow meters 62 and 74 are used to monitor the carrier and sparging flow rates.

Data Acquisition System 66: The data acquisition system 66 is used to collect data from detector, mass flow meter, temperature sensor, and pressure sensor and to control operation of the apparatus.

Breakthrough Detectors 88: The breakthrough detector can be an Electron Capture Detector (ECD) 88, which is very selective and sensitive to halogenated organic compounds, or a dry electrolytic conductivity detector, which is also very selective sensitive to chlorinated and brominated organic compounds. Both are designed for gas chromatography (GC) and require a low gas flow rate. The gas from the canister outlet is greater than 3 L/minute, so splitting the gas flow may be necessary to meet the maximum sensitivity requirement for either detector. The splitting 90 is achieved through the use of stainless steel restriction tubing. Examples of detectors 88 are Electron Capture Detector (ECD), Cat #, 8690-0020, SN: N6081. Dry Electrolytic Conductivity Detector (DELCD), Cat #, 8690-1026, from SRI Instruments.

Data Acquisition and Control System 66: The system includes four channel data collection and 8-TTL output for instrument control. An example is SN: N3531W, from SRI Instruments.

Prototype testing, breakthrough vs. remaining sorption capacity: Test data are given in Tables C and D.

TABLE C
Typical relative standard deviation, for the breakthrough time
96 determination during the fresh activated carbon tests.

| Activated | Add | Fresh | Fresh | Fresh | Fresh | Fresh | | RSD |
|---|---|---|---|---|---|---|---|---|
| carbon | DIW | 1 | 2 | 3 | 4 | 5 | Avg | (%) |
| BT (min) | 91.87 | 99.73 | 85.32 | 100.85 | 89.90 | 94.27 | 93.66 | 6.33 |

TABLE D
The relative standard deviation for the breakthrough time 96
determination under different fresh activated carbon levels.

| | | | | |
|---|---|---|---|---|
| Fresh Carbon (%) | 30 | 55.6 | 74.6 | 100 |
| RSD (%), n = 3 | 10.5 | 5.8 | 7.9 | 3.1 |

Test results indicated that up to 8.3% water loading did not significantly affect the breakthrough time. The 8.3% loading rate is relative to water completely condensed from 50% humidity of carrier gas during the testing period. Although water's boiling point (BP) is 100° C., our test results indicated that the condensed water will keep moving out of the activated carbon if dry gas is supplied continuously. After 3.5 hours, 54% of the water was washed out. In contrast, 2-chloroethyl methyl ether (2CEME)'s BP is 89° C., and it will be adsorbed by activated carbon tightly if not oversaturated. With >20-hours nitrogen elution, 2CEME was not washed out from the carbon. The comparison indicates that water can not be effectively adsorbed by the activated carbon to significantly decrease its adsorption capacity to organic compounds. Up to 50% humidity does not affect the adsorption capacity of activated carbon against most organic compounds.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A method to determine the remaining sorption capacity of an activated carbon filter comprising:
selecting a test gas;
performing a calibration procedure to generator a calibration graph (curve);
inserting a test filter into the gas flow circuit of the test apparatus with temperature control of the sparging gas generator, gas flow circuit, and test filter;
determining the breakthrough time; and
using the calibration curve to determine the remaining sorption capacity of the test filter.

2. The method of claim 1 wherein the test gas is selected to have a sufficiently low boiling point so that condensation of the test gas is avoided at the test operation temperature.

3. The method of claim 2 wherein the test gas is a halogenated compound.

4. The method of claim 2 wherein the test gas is halogenated but not fluorinated and the determination of breakthrough time is performed using a dry electrolytic conductivity detector.

5. The method of claim 2 wherein the test gas is selected to be dichloromethane.

6. The method of claim 2 wherein the mock contaminant compound is either one or a mixture of both of the group consisting of dimethyl methylphosphonate and 2-chloroethyl methyl ether.

7. The method of claim 2 wherein the calibration curve is generated by performing the step of adding a Mock Contaminant gas to the activated carbon by using a low toxicity organic compound to saturate a portion of the activated carbon and then performing the additional steps of:
mixing the said Mock Contaminant saturated carbon with the fresh activated carbon;
packing the mixture into an empty canister with a selected ratio of fresh and saturated carbon;
measuring the breakthrough time, so that the breakthrough time vs fraction of fresh carbon is determined; and subsequently
repeating the said mixing, packing, and measuring for a set of various ratios of fresh and saturated carbon; and optionally
repeating the above steps with additional selected test operation conditions selected from the group consisting of temperature, carrier gas flow rate, sparging gas flow rate, and breakthrough time range, to generate a set of calibration curves.

8. The method of claim 1, further comprising the step of:
correlating the results for the test canister to a main filter.

9. An apparatus for the determination of the remaining sorption capacity of an activated carbon filter by the method of claim 1, said apparatus comprising:
a carrier gas supply and a gas sparging gas generator that provides a selected test gas, said supply and generator having gas regulation and thermal and flow stabilization;
a test canister connection system that is a canister holder that permits easy emplacement of a test canister or connection means so that an isolated test canister can be inserted into the test gas/carrier gas flow system of the test apparatus;
a temperature control means for the test canister, gas supply, and test gas generator circuit;
a selective detector of the test gas; and
a data acquisition and instrument control system, which includes operating software and a computer with a display.

10. The apparatus of claim 9 wherein the selective detector is an electron capture device.

11. The apparatus of claim 9 wherein the selective detector is a dry electrolytic conductivity detector.

12. The apparatus of claim 9 wherein the test gas is dichloromethane.

13. The apparatus of claim 9 wherein the mock contaminant compound is either one or a mixture of both of the group consisting of dimethyl methylphosphonate and 2-chloroethyl methyl ether.

14. The apparatus of claim 9, further comprising:
a stable gas-split system.
